(19) United States
(12) Patent Application Publication
(10) Pub. No.: US 2006/0258692 A1
(43) Pub. Date: Nov. 16, 2006

(54) Quinazolinone compositions for regulation of gene expression related to pathological processes

(76) Inventors: Mark Pines, Rehovot (IL); Arnon Nagler, Jerusalem (IL); Shai Yarkoni, Kfar Sabe (IL); Israel Voldavksy, Mevaseret Zion (IL)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/533,371

(22) PCT Filed: Oct. 30, 2003
(86) PCT No.: PCT/IL03/00900

§ 371 Date: Jun. 1, 2006

Related U.S. Application Data
(60) Provisional application No. 60/422,487, filed on Oct. 31, 2002.

(51) Int. Cl. A61K 31/517

(52) U.S. Cl. 514/266.22

(57) ABSTRACT

The present invention relates to pharmaceutical compositions for modifying gene expression in a pathological process, thereby preventing or ameliorating said process. More particularly the compositions comprise quinazolinones, especially halofuginone, for inhibiting or preventing alterations in gene expression during fibrosis. The present invention particularly relates to pharmaceutical compositions for improving the regeneration of cirrhotic liver.

### FIELD OF THE INVENTION

The present invention relates to the field of regulation of mammalian gene expression by quinazolinone compositions and use thereof in treating mammalian disease. Specifically, the present invention relates to compositions comprising quinazolinones, especially halofuginone, for inhibiting or preventing alterations in gene expression induced during fibrosis. The present invention particularly relates to pharmaceutical compositions for improving the regeneration of cirrhotic liver.

### BACKGROUND OF THE INVENTION

Quinazolinones with Anti-Fibrotic Activity

U.S. Pat. No. 3,320,124 disclosed and claimed a method for treating coccidiosis with quinazolinone derivatives. Halofuginone, otherwise known as 7-bromo-6-chloro-3-[3-(3-hydroxy-2-piperidinyl)-2-oxopropyl]-4(3H)-quinazolinone (one of the quinazolinone derivatives), was first described and claimed in said patent by American Cyanamid company, and was the preferred compound taught by said patent and the one commercialized from among the derivatives described and claimed therein. Subsequently, U.S. Pat. Nos. 4,824,847; 4,855,299; 4,861,758 and 5,215,993 all related to the coccidiocidal properties of halofuginone.

More recently, U.S. Pat. No. 5,449,678 to some of the inventors of the present invention discloses that these quinazolinone derivatives are unexpectedly useful for the treatment of a fibrotic condition. This disclosure provides compositions of a specific inhibitor comprising a therapeutically effective amount of a pharmaceutically active compound of the general formula:

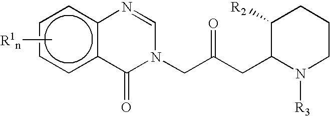

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

U.S. Pat. No. 5,449,678 discloses that these compounds are effective in the treatment of fibrotic conditions such as scieroderma and graft versus host disease (GVHD). U.S. Pat. No. 5,891,879 further discloses that these compounds are effective in treating restenosis. Fibrosis and restenosis are associated with excessive collagen deposition, which can be inhibited by halofuginone. Restenosis is characterized by smooth muscle cell proliferation and extracellular matrix accumulation within the lumen of affected blood vessels in response to a vascular injury. One hallmark of such smooth muscle cell proliferation is a phenotypic alteration, from the normal contractile phenotype to a synthetic one. Type I collagen has been shown to support such a phenotypic alteration, which can be blocked by halofuginone (Choi E. T. et al., 1995. Arch. Surg., 130: 257-261; U.S. Pat. No. 5,449,678).

Notably, halofuginone inhibits collagen synthesis by fibroblasts in vitro; however, it promotes wound healing in vivo (WO 01/17531). Halofuginone was also shown to have different in vitro and in vivo effects on collagen synthesis in bone chondrocytes. As discussed in U.S. Pat. No. 5,449,678 halofuginone inhibits the synthesis of collagen type I by bone chondrocytes in vitro. However, chickens treated with halofuginone were not reported to have an increased rate of bone breakage, indicating that the effect is not seen in vivo. Thus, the exact behavior of halofuginone in vivo cannot always be accurately predicted from in vitro studies.

Quinazolinone-containing pharmaceutical compositions, including halofuginone, have been disclosed and claimed as effective for treating malignancies (U.S. Pat. No. 6,028,075), for prevention of neovascularization (U.S. Pat. No. 6,090,814), as well as for treating hepatic fibrosis (U.S. Pat. No. 6,562,829).

Halofuginone and Gene Expression

In accordance with the activity of halofuginone as an inhibitor of collagen type I synthesis, halofuginone has been found to inhibit the gene expression of collagen type α1(I) but not of type II or type III. In culture, halofuginone attenuated collagen α1(I) gene expression and collagen production by murine, avian and human skin fibroblasts, derived from either scleroderma or chronic graft-versus-host disease (cGVHD) patients. In animal models of fibrosis in which excess collagen is the hallmark of the disease, administration of halofuginone prevented the increase in collagen synthesis and collagen α1(I) gene expression. These models included mice afflicted with cGVHD and tight skin (Tsk+) mice (Levi-Schaffer F. et al., 1996. J Invest Dermatol. 106:84-88; Pines M. et al., 2001 Biochem Pharmacol 62:1221-1227), rats with pulmonary fibrosis after bleomycin treatment (Nagler A. et al., 1996. Am J Respir Crit Care Med. 154:1082-1086) and rats that developed adhesions at various sites (Nyska M. et al., 1996. Connect Tissue Res. 34:97-103). The inventors of the present invention and coworkers have previously reported that topical treatment of a cGVHD patient with halofuginone caused a transient attenuation of collagen α1(I) gene expression, thus demonstrating human clinical efficacy.

International Patent Application WO 00/09070 discloses that halofuginone and related quinazolinones inhibit not only the synthesis and gene expression of collage type I, but a cascade of pathogenic processes initiated by trauma. Specifically, halofuginone was found to regulate the extracellular cell matrix economy at the molecular level. The present invention relates to pharmaceutical compositions for improving the regeneration of fibrotic liver. It has been previously demonstrated that various agents which regulated gene expression in liver cells in vitro were not always similarly active under physiological activation in vivo. This phenomenon is probably partly due to the lack of cellular heterogeneity in the in vitro examined culture.

Quinazolinones and Hepatic Cirrhosis and Regeneration

Fibrosis represents the response of the liver to diverse chronic insults such as chronic viral infection, alcohol, immunological attack, hereditary metal overload, parasitic diseases, and toxic damage. Because of the worldwide prevalence of these insults, liver fibrosis is common and ultimately culminates in cirrhosis which is associated with significant morbidity and mortality. Hepatic fibrosis, regardless of the cause, is characterized by an increase in extracellular matrix (ECM) constituents, although their relative distribution within the liver lobule varies with the site and nature of the insult (George J. et al., 1999. PNAS USA 96:12719-12724).

In the injured liver, the hepatic stellate cells (HSC) constitute the major source of the ECM. These cells are usually quiescent with a low proliferation rate but, upon activation, probably because of hepatocyte injury they differentiate into myofibroblast-like cells, with high proliferative capacity. The predominant ECM protein synthesized by the HSCs in fibrosis is collagen type I, primarily because of increased transcription of the type I collagen genes. Increase in the gene expression of other types of collagens such as types III and IV as well as of other matrix proteins have also been reported. Liver fibrosis may also result from a relative imbalance between production and degradation of matrix proteins. Activated HSC constitute the source of various collagenases and tissue inhibitors of metalloproteinases (TIMPs) required for ECM remodelling (Iredale J P. et al., 1996. Hepatology 24:176-184; Arthur M J. et al., 1998. J Gastroenterol Hepatol 13:S33-S38).

U.S. Pat. No. 6,562,829 to some of the inventors of the present invention discloses that halofuginone inhibits the pathophysiological process of hepatic fibrosis in vivo, possibly by inhibiting collagen type I synthesis. Halofuginone has been shown to prevent HSC activation and abolish the increase in collagen α1(I) gene expression and collagen deposition in rats insulted with either dimethylnitrosamine (DMN), or thioacetamide (TAA). When given to rats with established fibrosis, halofuginone caused almost complete resolution of the fibrotic condition.

There is now a substantial body of evidence, derived from both animal models and human liver diseases, to indicate that liver fibrosis and cirrhosis are dynamic processes that can both progress and regress over time. Both the progression and resolution of the fibrotic lesions requires cellular cross talk of various cell types populating the liver.

Liver regeneration after the loss of hepatic tissue is a fundamental parameter of liver response to injury. This long-time recognized phenomenon is now defined as a coordinated response induced by specific external stimuli and involving sequential changes in gene expression, growth factor production, and morphologic structure. Many growth factors and cytokines, most notably hepatocyte growth factor, epidermal growth factor, transforming growth factor-α, interleukin-6, tumor necrosis factor-α, insulin, and norepinephrine, appear to play important roles in this process. In IL6 −/−mice, a highly significant reduction in hepatocyte DNA synthesis, increased liver necrosis, discrete G₁-phase abnormalities including absence of STAT3 activation, reduction in AP-I activation, and selective abnormalities in gene expression are observed post-hepatectomy and after carbon tetrachloride injury, all of which are corrected by injection with IL-6. Among those genes whose expression is abnormal in IL6−/−liver after partial hepatectomy are those encoding protein involved in cell cycle progression such as AP-I factor, c-Myc, and cyclin D1. However, a number of other genes with less clear connection to cell growth show blunted induction in the absence of IL-6, including the insulin-like growth factor binding protein-1 (IGFBP-1) gene.

Liver regeneration involves proliferation of mature, functioning cells composing the intact organ. Following toxic damage, hepatitis, surgical resection and the like a renewal system may be induced. The induction leads to the proliferation of parenchymal cells which are normally in Go, resulting in the restoration of the hepatic parenchyma.

Post-hepatectomy liver insufficiency is one of the main problems associated with major hepatic resection. This is especially true in cirrhotic livers that have reduced functional reserve. In hepatocellular carcinoma, which is often associated with cirrhosis, extensive resection to prevent occurrence of malignant tumors is a questionable procedure, as in cirrhotic liver regeneration is impaired. Improving the regeneration capacity of a damage liver would therefore enable better treatment of hepatocellular carcinoma. Preliminary results of the inventors of the present invention and co-workers (Spira G. et al., 2002. J. Hepatol. 37: 331-339) showed that halofuginone improved the capacity of cirrhotic liver to regenerate after partial hepatectomy. Treatment of an existing pathological condition is most often the desired therapy, as preventive therapeutic regimes are often less applicable. Treatment of liver tissues after the damage has already occurred by improving liver regeneration would be therefore highly beneficial.

Thus, there is a recognized unmet medical need for effectors capable of improving liver regeneration. It would be highly advantageous to have such effectors that intervene at the transcriptional or other molecular level, such that the effect would not interfere with any other beneficial repair mechanisms.

### SUMMARY OF INVENTION

The present invention related to pharmaceutical compositions for improving the regeneration of fibrotic tissues. Specifically, the present invention is directed to pharmaceutical compositions for modifying gene expression in a pathological process, thereby preventing or ameliorating said pathological process. In a first aspect the present invention is directed at pharmaceutical compositions for improving the regeneration of a fibrotic liver. In a second aspect the present invention is directed to pharmaceutical compositions for modifying gene expression that is involved in fibrosis. In a third aspect the present invention is directed to pharmaceutical compositions for modifying gene expression induced by a toxin or toxic substance, thereby preventing or ameliorating the pathological process induced by said toxin.

Unexpectedly, it has been found, as described herein below, that halofuginone improves the regeneration of thioacetamide (TAA) induced cirrhotic liver after partial hepatectomy. Halofuginone prevents thioacetamide (TAA) dependent alteration in gene expression, specifically the regulation of insulin like growth factor binding protein 1 (IGFBP-1) gene. Without wishing to be bound by any theory or any mechanism, the prevention of the TAA-induced down-regulation of the IGFBP-1 gene by halofuginone may explain the resolution of liver fibrosis observed after halofuginone treatment and the beneficial effect of halofuginone on cirrhotic liver regeneration.

According to one aspect, the present invention provides methods for improving the regeneration capacity of a cirrhotic liver.

According to one embodiment, the present invention provides a method for improving liver regeneration comprising administering to an individual in need thereof a pharmaceutical composition comprising a therapeutically effective amount of a compound having the formula:

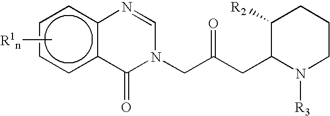

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for improving liver regeneration.

According to another aspect the present invention provides methods for treating or preventing pathological processes related to alteration in gene expression during fibrosis.

According to one embodiment, the present invention provides methods for treating or preventing pathological processes related to alteration in gene expression due to fibrotic processes, comprising administering to an individual in need thereof of a pharmaceutical composition comprising a therapeutically effective amount of a compound having the formula:

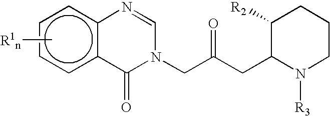

wherein: n=1 -2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

According to certain embodiments the fibrotic process is liver fibrosis.

According to another aspect the present invention provides methods for preventing alterations in gene expression due to exposure to a toxin.

According to one embodiment the present invention provides methods for preventing alterations in gene expression due to exposure to a toxin comprising administering to an individual in need thereof a pharmaceutical composition comprising a therapeutically effective amount of a compound having the formula:

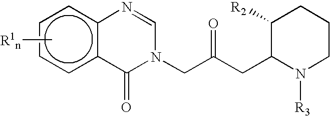

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

According to a certain embodiment the toxin is thioacetamide (TAA), which is known to induce fibrotic changes in hepatocytes.

According to yet another embodiment compositions of the present invention alter the expression of at least one gene selected from the group consisting of:

IGFBP-1—Insulin like growth factor binding protein 1

IGFBP-3—Insulin like growth factor binding protein 3

PRL-1 (or PTP4A1)—protein tyrosine phosphatase 4A1

APO-AIV—Apolipoprotein A—IV precursor

PI 3-kinase p85-alpha subunit

MAP kinase p38—Mitogen activated protein kinase p38

Proteasome component C8

E-FABP (FABP5 or C-FABP)—Epidermal fatty acid-binding protein

PMP-22 (SR13 myelin protein)—peripheral myelin protein 22

PCNA—proliferation cell nuclear antigen

Proteasome activator rPA28 subunit alpha

c-K-ras 2b proto-oncogene

ST2A2—Alcohol sulfotransferase A, Probable alcohol sulfotransferase

TIMP-2—Metalloproteinase inhibitor 2 (Precursor), Tissue inhibitor of metalloproteinase 2

MMP-3—metalloproteinase 3

MMP-13—metalloproteinase 13

According to another embodiment the compositions are used to modify expression of a gene wherein the gene is a member of the IGFBP family. According to another embodiment the gene is IGFBP-1. According to yet another embodiment the gene is IGFBP-3.

According to another embodiment the present invention provides a method for treatment or prevention of hepatic cirrhosis by increasing IGFBP-1 gene expression in hepatocytes comprising administering a pharmaceutical composition comprising a therapeutically effective amount of a compound having the formula:

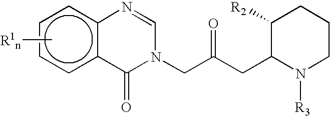

wherein: n=1 -2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

According to another embodiment the present invention provides a method for improving the capacity of a cirrhotic liver to regenerate following partial hepatectomy by inducing the expression of at least one gene selected from the group of IGFBP-1, PRL-1, MMP-3 and MMP-13 comprising administering a pharmaceutical composition comprising a therapeutically effective amount of a compound having the formula:

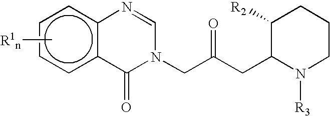

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

According to another embodiment the present invention provides a method for improving the capacity of a cirrhotic liver to regenerate following partial hepatectomy by affecting the molecules in the signal transduction pathway of hepatocyte growth factor (HGF), comprising administering a pharmaceutical composition comprising a therapeutically effective amount of a compound having the formula:

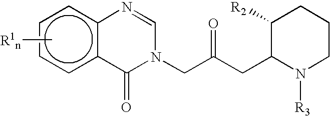

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl and pharmaceutically acceptable salts thereof.

Of this group of compounds, halofuginone has been found to be particularly effective for such treatment.

According to another embodiment of the present invention the compositions comprising quinazolinones and especially halofuginone are useful for enhancing the amount of biologically active IGF-1.

### BRIEF DESCRIPTION OF THE DRAWINGS

FIG. 1 shows histological analysis of liver sections. Liver samples were taken from control rats, rats treated with halofuginone (H, 5 ppm in the diet), TAA (T, 200 mg/kg twice weekly) or a combination of the two for 4 weeks (T+H). The sections were stained with hematoxylin and eosin (H&E), and with Sirius red for collagen. Stellate cells and TIMP-II were detected by immunohistochemistry. Collagen α1(I) gene expression was evaluated by in situ hybridization. Note the high levels of alpha smooth-muscle actin (αSMA)-positive stellate cells that express the collagen α1(I) gene and synthesize collagen and tissue inhibitors of metalloproteinases II (TIMP-II) after TAA treatment. A marked resolution of the fibrotic lesion was observed with halofuginone.

FIG. 2 Liver regeneration of healthy and TAA treated rats with or without subsequent halofuginone diet. Rats underwent 70% partial hepatectomy for 48 hours at which time the animals were sacrificed. Restituted liver mass (FIG. 2A) and PCNA labeling index (FIG. 2B) monitored the capacity of the liver to regenerate. PCNA labeling index was scored at the time of surgery and after 48 hours. The beneficial usage of halofuginone is demonstrated by an improved capacity to regenerate.

FIG. 3 describes the effect of halofuginone on rat liver gene expression. Total RNA from liver tissue was hybridized with Atlas microarray filters. FIG. 3A—Microarray analysis of liver biopsies of rats treated for 4 weeks with TAA alone (200 mg/kg twice weekly). FIG. 3B—Microarray analysis of liver biopsies of rats treated for 4 weeks with TAA (200 mg/kg twice weekly) in combination with halofuginone (5 ppm in the diet). The arrows point to the differentially expressed genes. FIG. 3C—Expression of PRL-1 and ApoA-IV. Total RNA was prepared from liver biopsies of rats treated with TAA (T) and in combination with halofuginone (T+H). Ribosomal 28S RNA was used as the directive of RNA loading.

FIG. 4 shows the increase in IGFBP-1 gene expression elicited by halofuginone in vivo. IGFBP-1 gene expression was evaluated by Northern blots (FIG. 3A) and by in situ hybridization (FIG. 3B). FIG. 4A—Total RNA was prepared from liver biopsies of the control rats (C), rats treated with TAA (T) and halofuginone alone (H) or in combination (T+H) after 1, 2 and 4 weeks and hybridized with the IGFBP-1 or IGFBP-3 probes. Ribosomal 18S RNA was used as the directive of RNA loading. FIG. 4B—Sections of livers after 4 weeks of treatment were hybridized with the IGFBP-1 probe. Dark-field photomicrographs showing hybridization of antisense IGFBP-1 probe to liver sections of control rats (C), rats treated with TAA (T) and rats treated with a combination of TAA and halofuginone (T+H). Hybridization with sense IGFBP-1 probe was used as a negative control.

FIG. 5 shows the effect of halofuginone on IGFBP-1 synthesis in various cell types. FIG. 5A—HepG2, Huh-7, Hep3B, Det551, ROS and HSC cells were incubated with and without 50 nM halofuginone in a serum-free medium. The IGFBP-1 gene expression was analyzed by Northern blotting (NB) and the presence of IGFBP-1 in the condition medium was evaluated by Western blotting (WB). Note that only hepatocytes synthesized IGFBP-1 in response to halofuginone. FIG. 5B—Rat primary hepatocytes were incubated with insulin (Ins, 100 nM) or halofuginone (Halo, 1 nM) for 24 h and IGFBP-1 was detected by Western blot.

FIG. 6 shows the effect of halofuginone on IGFBP-1 synthesis and cell proliferation: dose and time response. HepG2 cells were incubated with various concentrations of halofuginone for 24 h and the level of IGFBP-1 gene expression was analyzed by Northern blotting (FIG. 6A) and the content of IGFBP-1 in the condition medium was evaluated by Western blotting (FIG. 6B). FIG. 6C and FIG. 6D represent the levels of IGFBP-1 gene expression and of IGFBP-1 in the condition medium and in response to 50 nM halofuginone after various intervals, respectively. FIG. 6E—Cells were incubated for 24 h with various concentrations of halofuginone. The results represented as the mean cell number±SE of 6 replicates.

FIG. 7 describes the effect of Cyclohexamide on IGFBP-1 up regulation by halofuginone. FIG. 7A—Following serum starvation, HepG2 cells were incubated with 50 nM halofuginone for the indicated time after which the medium was replaced with fresh medium without halofuginone. The level of IGFBP-1 was evaluated by Western blotting 24 h after the beginning of the experiment. FIG. 7B—HepG2 cells were incubated for 24 h with and without 10 μg/ml cyclohexamide (CX) and 50 nM halofuginone. Expression of IGFBP-1 was analyzed by Northern blotting.

FIG. 8 shows the inhibition of stellate cell motility by IGFBP-1. FIG. 8A—Hepatocytes (HepG2) conditioned medium after halofuginone treatment contained higher levels of IGFBP-1 compare to the control (Insert; lane 1−no halofuginone; lane 2+halofuginone). When added to stellate cells (HSC-T6), inhibition in cell motility was observed. Each time point represents the mean track area of 3-5 cells±S.E. FIG. 8B—Hepatocytes (HepG2) conditioned medium after halofuginone treatment was immunoprecipitated with anti IGFBP-1 antibodies or with normal goat serum and incubated with the stellate cells for motility evaluation. The media were added for 8 h to the stellate cells. Each column represents the mean track area of 10-20 cells±S.E. The level of IGFBP-1 in the media before and after the immunoprecipitation is described in the insert. Lane 1—no halofuginone treatment; lane 2—after halofuginone treatment; lane 3—medium after immunoprecipitation with anti IGFBP-1 antibodies; lane 4—IGFBP-1 in the precipitate after treatment with the anti IGFBP-1 antibodies.

### DETAILED DESCRIPTION OF PREFERRED EMBODIMENTS

The anti-fibrotic activity of certain quinazolinones has been demonstrated in various systems. Among these compounds a particularly preferred embodiment is halofuginone.

The mechanism of action of these compounds has previously been the subject of some speculation and gene expression induced by exposure to the compound was studied, and it was found that halofuginone inhibited expression of various subtypes of collagen and other matrix proteins. Nevertheless, at the same time healing processes were not impaired or inhibited. In fact, the contrary was observed, and healing processes were improved by treatment with halofuginone.

The present invention relates to pharmaceutical compositions for improving the regeneration of fibrotic liver. The present invention further relates to genes which are differentially expressed due to the presence of halofuginone. More specifically, the present invention relates to the differential expression of genes in fibrotic tissues treated with halofuginone. The present invention further relates to the differential expression of genes in tissues exposed to a toxin and treated with halofuginone. Advantageously, the methods provided by the present invention enable the elucidation of the in vivo effect of halofuginone on the differential gene expression during fibrosis.

It is now disclosed for the first time that halofuginone enhances the processes involved in growth and regeneration of damaged fibrotic tissues. The beneficial effects of halofuginone may be due to the fact that it increases the availability or activity of Insulin Growth Factor-1.

Unexpectedly it has now been found, as described herein below, that compounds having the formula:

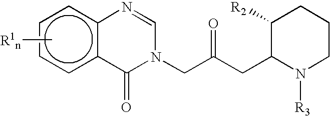

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl; and pharmaceutically acceptable salts thereof, improve the regeneration capacity of fibrotic tissues, specifically the regeneration capacity of fibrotic liver.

Of this group of compounds, halofuginone having the formula: 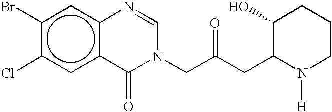
has been found to be particularly effective.

As used herein, the term “lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like. The term “alkenyl” refers to a group having at least one carbon-to-carbon double bond.

The terms “alkoxy” and “alkenoxy” denotes —OR, wherein R is alkyl or alkenyl, respectively.

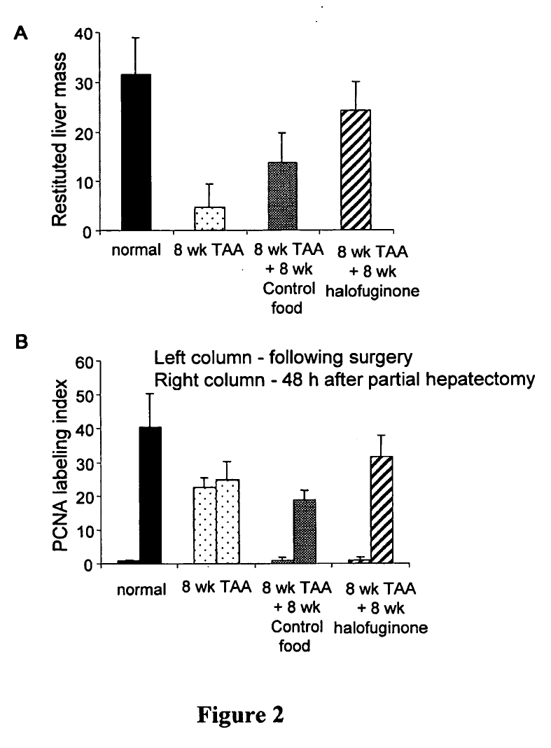

TAA is used as a model for liver fibrosis. When administered by intraperitoneal (i.p.) injection, TAA induces liver cirrhosis, including the deposition of fibrotic tissues and the loss of liver function. The inventors of the present invention and co-workers have previously shown (U.S. Pat. No. 6,562,829) that in rats treated with dimethylnitrosamine or TAA, halofuginone prevents stellate cell (HSC) activation in the liver and abolishes the increase in collagen α-1(I) gene expression and collagen deposition. In addition, halofuginone markedly improved the capacity of cirrhotic liver to regenerate after partial hepatectomy, as described by Spira et al., (supra), the content of which is hereby fully incorporated by reference. Halofuginone treatment significantly improved liver regeneration demonstrated by an increase in restituted liver mass (FIG. 2A) and PCNA labeling index (FIG. 2B). The improved regeneration was also reflected by the reduction in the number of αSMA-positive cells, reduction in collagen and TIMP-2 content and improvement in Ishak staging.

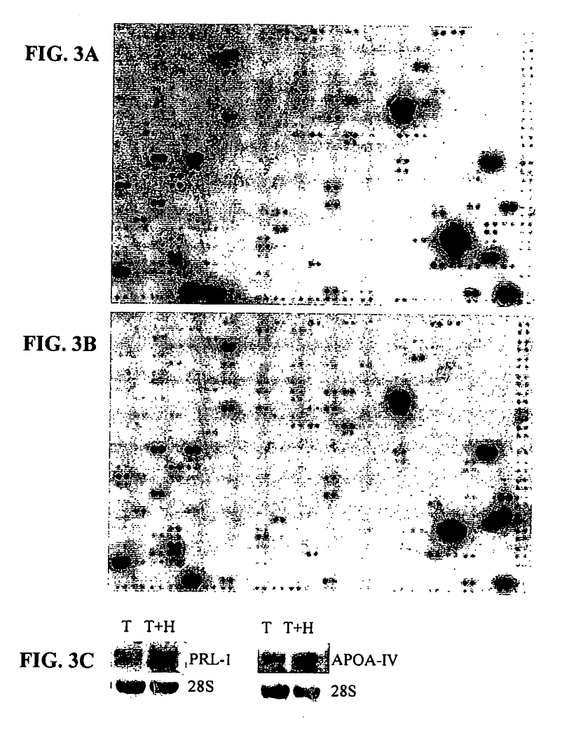

Hepatic fibrosis/cirrhosis is characterized by excessive production of ECM by activated HSC due to collagen synthesis and inhibition of collagen degradation. Thus, pharmacological intervention to treat liver fibrosis should, at least in part, aim to inhibit HSC activation, to inhibit ECM synthesis and/or to stimulate matrix protein degradation. To reverse cirrhosis, inhibition of collagen synthesis by activated HSC and normal functionality of hepatocytes and other cell types is essential. In a first attempt to identify genes responsible for halofuginone action in vivo, we compared gene pattern of livers with Ishak grade 5-6 with those with grade 1-2 after halofuginone treatment (FIGS. 3 A&B). Of the 588 genes of the array, 13 were differentially expressed.

According to another aspect, the present invention provides methods for treating and preventing pathological processes related to alteration in gene expression during fibrotic processes.

According to one embodiment of the present invention, halofuginone prevented alteration in gene expression during fibrosis wherein the genes are selected from the group consisting of:

IGFBP-1—Insulin like growth factor binding protein 1

IGFBP-3—Insulin like growth factor binding protein 3

PRL-1 (or PTP4A1)—protein tyrosine phosphatase 4A1

APO-AIV—Apolipoprotein A-IV precursor

PI 3-kinase p85-alpha subunit

MAP kinase p38—Mitogen activated protein kinase p38

Proteasome component C8

E-FABP (FABP5 or C-FABP)—Epidermal fatty acid-binding protein

PMP-22 (SR13 myelin protein)—peripheral myelin protein 22

PCNA—proliferation cell nuclear antigen

Proteasome activator rPA28 subunit alpha

c-K-ras 2b proto-oncogene

ST2A2—Alcohol sulfotransferase A, Probable alcohol sulfotransferase

TIMP-2—Metalloproteinase inhibitor 2 (Precursor), Tissue inhibitor of metalloproteinase 2

MMP-3—metalloproteinase 3

MMP-13—metalloproteinase 13

According to another embodiment of the present invention, halofuginone prevented alteration in gene expression during fibrosis wherein the genes are selected from the IGFBP family.

According to yet another embodiment of the present invention, halofuginone prevented alteration in gene expression during fibrosis wherein the genes are IGFBP-1 and IGFBP-3.

According to one embodiment, halofuginone prevented alteration in gene expression during liver fibrosis.

The present invention now discloses that halofuginone prevents the TAA-induced down-regulation of the IGFBP-1 gene that may explain the resolution of liver fibrosis observed after halofuginone treatment and the beneficial effect of halofuginone on cirrhotic liver regeneration.

In addition, in the present invention we focused our attention on the effect of halofuginone on IGFBP synthesis, because of the involvement of the IGF-1 axis in liver physiology in health and disease. In fibrosis/cirrhosis major alterations in the GH/IGF-I axis were observed including local changes in the expression of the genes encoding different members of the IGFBP family and changes in the plasma levels of IGF-I and its binding proteins. In liver fibrosis, a poor correlation between the expression of the IGFBPs genes and their plasma concentrations has been observed, which may reflect an alteration in their clearance.

IGFBP-1 is an immediate-early gene induced at the transcriptional level in the remnant liver following partial hepatectomy, or after any other liver-damaging processes that result in liver regeneration. It is distinct in that its plasma level is dynamically regulated by changes in the metabolic state and after hepatic injury. The IGFBP-1 promoter has been extensively studied. Traditional promoter and deletion analyses indicate that highly conserved sequences within a few hundred bases upstream of the transcription initiation site confer liver specific and hormonal regulation. DNase I hypersensitivity analyses identified clusters of liver-restricted nuclear sensitive sites in the promoter region. This tissue-specific pattern of expression may be regulated in part by members of hepatocyte nuclear factor (HNF-1) family of protein, as the HNF-1 forms are responsible for the basal IGFBP-1 promoter activity in hepatoma cells via a conserved site just upstream of the RNA initiation site.

IGFBP-3, synthesized by Kupffer and endothelial cells is the most abundant circulating IGFBP in adult mammalian species including rats and humans. IGF-I, IGFBP-3 and an acid labile subunit form a 150-kDa ternary complex that prolongs the plasma half-life of IGF-I and limits the amounts of free, biologically active IGF-I in circulation. IGF-I also circulates bound to other IGFBPs, but their physiological significance is less well established.

According to one embodiment the present invention provides a method for the treatment of hepatic cirrhosis by preventing down regulation of the IGFBP-1 expression in hepatocyte cells by administering a pharmaceutically effective amount of a compound having the formula:

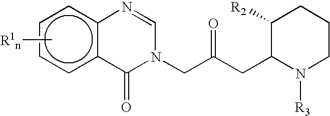

wherein: n=1 -2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl. Pharmaceutically acceptable salts thereof are also included.

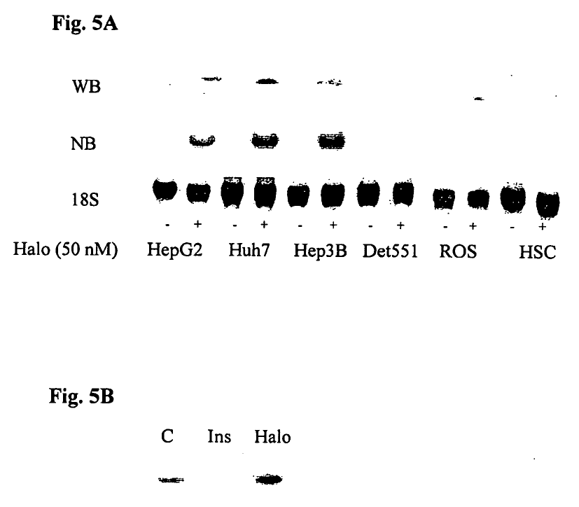

Halofuginone affected IGFBP-1 synthesis exclusively in hepatocytes (FIG. 5), which was consistent with the notion of hepatocytes being the major source of IGFBP-1 in the liver.

According to another aspect the present invention provides methods for preventing alterations in gene expression due to exposure to a toxin. According to one embodiment the present invention provides methods for preventing alterations in gene expression due to exposure to a toxin comprising administering to an individual in need thereof a pharmaceutical composition comprising a therapeutically effective amount of a compound having the formula:

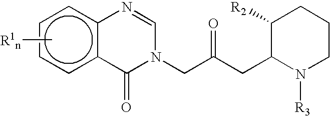

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl, and pharmaceutically acceptable salts thereof.

Of this group of compounds, halofuginone has been found to be particularly effective for such treatment.

According to a certain embodiment the toxin is thioacetamide (TAA), which is known to induce fibrotic changes in hepatocytes.

According to yet another aspect, the present invention provides methods for improving the regeneration of an injured liver by treating or preventing pathological processes related to alteration in gene expression during liver fibrosis.

According to one embodiment the present invention provides a method for improving the capacity of a cirrhotic liver to regenerate following partial hepatectomy by inducing the IGFBP-1 and PRL-1 gene expression by administering a pharmaceutically effective amount of a compound having the formula:

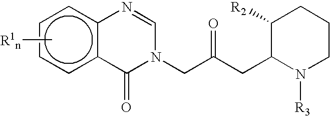

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl. Pharmaceutically acceptable salts thereof are also included.

Of this group of compounds, halofuginone has been found to be particularly effective in such treatment.

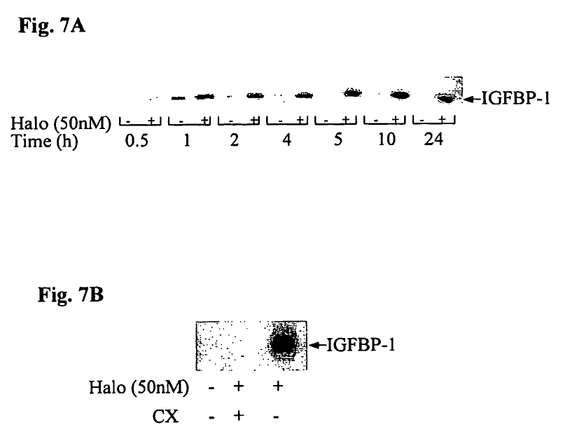

Two of the immediate-early genes that are induced at the transcriptional level in the remnant liver following partial hepatectomy, and which are probably important in maintaining hepatic metabolism during regeneration are IGFBP-1 and the protein tyrosine phosphatase 4A1 (PRL-1). Both of these genes were up regulated by halofuginone (FIG. 3). This observation could account for the immense improvement in the capacity of a cirrhotic liver to regenerate after halofuginone treatment. In the regenerated liver, IGFBP-1 is regulated by interleukin 6 via hepatocyte nuclear factor 1 and induced factors STAT3 and activator protein 1 (AP-1, c-Fos/c-Jun). The inhibitory effect of halofuginone on collagen type I synthesis was also c-Jun dependent (Fan S. et al., 2000. Oncogene 19:2212-2223) raising the possibility that the same pathway is involved in halofuginone-dependent increase in the synthesis of IGFBP-1. Cyclohexamide annulled both the halofuginone-dependent activation of IGFBP-1 synthesis (FIG. 7) and the inhibition of collagen α1(I) gene expression (Halevy O. et al., 1996. Biochem Pharmacol 52:1057-1063) suggesting that de novo protein synthesis is prerequisite for halofuginone signal transduction.

According to another embodiment the present invention provides method for improving the capacity of a cirrhotic liver to regenerate following partial hepatectomy by affecting the molecules in the signal transduction pathway of hepatocyte growth factor, by administering a pharmaceutically effective amount of a compound having the formula:

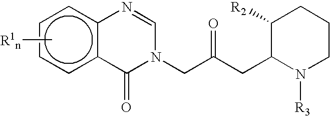

wherein: n=1-2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl. Pharmaceutically acceptable salts thereof are also included.

Of this group of compounds, halofuginone has been found to be particularly effective in such treatment.

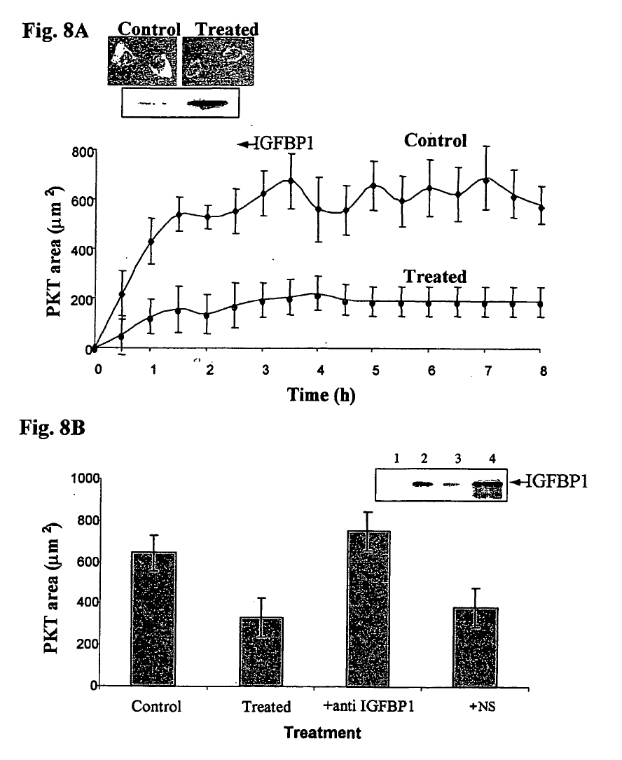

Phosphatidylinositol 3′kinase (PI3K) has been implicated in regulation of the IGFBP-1 gene in hepatocytes (Cichy S B. et al., 1998. J Biol Chem 273:6482-6487) and of the collagen type I gene in stellate cells (Svegliati-Baroni G. et al., 1999. Hepatology 29:1743-1751). Interestingly, the p85 α-subunit of the P13K was one of the battery genes up regulated by halofuginone. MAP kinase p38 was also up regulated by halofuginone, suggesting involvement of more than one pathway. It is interesting to note that hepatocyte growth factor (HGF) which has been shown to signal through PI3K accelerated liver regeneration after partial hepatectomy, decreased collagen synthesis in the TAA model of cirrhosis and induced IGFBP-1 gene expression. In addition to modulation of the IGF-1 bioavailability and action, IGFBP-1 has been implicated in other activities. IGFBP-1 has been implicated in inhibition of collagen type I gene expression directly, as well as by inhibiting the IGF-I-dependent collagen type I synthesis by stellate cells. IGFBP-1 has been also shown to regulate mitogenic signal pathways and to function as a critical hepatic survival factor in the liver by reducing the level of pro-apoptotic signals. Additional characteristic of IGFBP-1 is its ability to affect cell motility. The IGFBP-1 secreted by the HepG2 after halofuginone treatment inhibited stellate cell motility (FIG. 8). Stellate cell motility is dependent on collagen type I; thus in vivo, halofuginone may inhibit Stellate cell motility directly by inhibiting collagen type I production and by stimulating IGFBP-1 synthesis by hepatocytes causing a further inhibition in Stellate cell motility. This is of a major importance since migration capacity is part of the “activated” phenotype of stellate cells.

The compositions of the present invention may be administered by any means that can affect regulation of gene expression. For example, administration may be parenteral, subcutaneous, intravenous, intramuscular, intrathecal, oral, or topical.

While it is possible for the active ingredients to be administered alone, it is preferable to present them as pharmaceutical formulations. The formulations of the present invention comprise at least one active ingredient, as above defined, together with one or more acceptable carriers thereof and, optionally, other therapeutic ingredients. The carrier(s) must be acceptable in the sense of being compatible with the other ingredients of the formulation, and not deleterious to the recipient thereof.

The formulations may conveniently be presented in unit dosage form, and may be prepared by any of the methods well known in the art of pharmacy. Such methods include the step of bringing into association the active ingredient with the carrier, which constitutes one or more accessory ingredients. In general, the formulations are prepared by uniformly and intimately bringing into association the active ingredient with liquid carriers or finely-divided solid carriers, or both, and then, if necessary, shaping the product. The dosage of active ingredients in the composition of this invention may be varied; the selected form depends upon the route of administration, and on the duration of the treatment. Administration dosage and frequency will depend on the age and general health condition of the patient, taking into consideration the possibility of side effects. Administration will also be dependent on concurrent treatment with other drugs and the patient's tolerance of the administered drug.

Solid forms for oral administration include capsules, tablets, pills, powders and granules. In such solid forms, the active compound is admixed with at least one inert diluent, such as sucrose, lactose or starch. Such oral forms can also comprise, additional substances other than inert diluent. In the case of capsules, tablets and pills, the formulation may also comprise buffering agents. Tablets and pills can additionally be prepared with an enteric coating.

Liquid forms for oral administration include pharmaceutically acceptable emulsions, solutions, suspensions, syrups and elixirs, containing inert diluents commonly used in the pharmaceutical art. Besides inert diluents, such compositions can also include adjuvants, such as wetting agents, emulsifying and suspending agents, and sweeteners.

Preparations according to the present invention for parenteral administration include sterile aqueous or non-aqueous solutions, suspensions or emulsions. Examples of non-aqueous solvents or vehicles are propylene glycol, polyethylene glycol, vegetable oils such as olive oil, and injectable organic esters, such as ethyl oleate.

Topical administration can be effected by any method commonly known to those skilled in the art and include, but is not limited to, incorporation of the composition into creams, ointments, or transdermal patches. When formulated in a cream, the active ingredients may be employed with an oil-in-water cream base. If desired, the aqueous phase of the cream base may include, for example, at least 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active ingredient through the skin or other affected areas. Examples of such dermal penetration enhancers include dimethylsulphoxide and related analogues.

The oily phase of the emulsions of the present invention may be constituted from known ingredients in a known manner. While the phase may comprise merely an emulsifier (otherwise known as an emulgent), it desirably comprises a mixture of at least one emulsifier with a fat or an oil, or with both a fat and an oil. Preferably, a hydrophilic emulsifier is included, together with a lipophilic emulsifier, which acts as a stabilizer. It is also preferred to include both an oil and a fat. Together, the emulsifier(s), with or without stabilizer(s), make up the so-called emulsifying wax, and the wax, together with the oil and/or fat, make up the so-called emulsifying ointment base, which forms the oily dispersed phase of the cream formulations. Emulgents and emulsion stabilizers suitable for use in the formulation of the present invention include Tween 60, Span 80, cetostearyl alcohol, myristyl alcohol, glyceryl mono-stearate and sodium lauryl sulfate.

Although the specific quinazolinone derivative “halofuginone” is referred to throughout the specification, it is understood that other quinazolinone derivatives may be used in its place, these derivatives having the general formula:

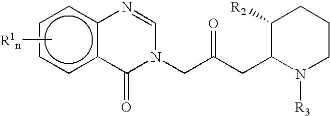

wherein: n=l -2

R₁ is at each occurrence independently selected from the group consisting of hydrogen, halogen, nitro, benzo, lower alkyl, phenyl and lower alkoxy;

R₂ is a member of the group consisting of hydroxy, acetoxy and lower alkoxy; and

R₃ is a member of the group consisting of hydrogen and lower alkenoxy-carbonyl. Pharmaceutically acceptable salts thereof are also included.

While the invention will now be described in connection with certain preferred embodiments in the following figures and examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following figures and examples which include preferred embodiments will serve to illustrate the practice of this invention, it being understood that the particulars shown are by way of example and for purposes of illustrative discussion of preferred embodiments of the present invention only, and are presented in the cause of providing what is believed to be the most useful and readily understood description of formulation procedures as well as of the principles and conceptual aspects of the invention.

### EXAMPLES

Materials

Halofuginone bromhydrate was from Collgard Biopharmaceuticals Ltd (Tel Aviv, Israel). TAA was from Sigma (St Louis, Mo., USA). Alpha smooth-muscle actin (αSMA) monoclonal antibodies (1:200 dilution) were from Dako A/S (Glostrup, Denmark). TIMP-2 polyclonal antibodies (1:50 dilution) and the Histomouse SP kit (second antibodies) were from Zymed Laboratories Inc. (South San Francisco, Calif., USA). IGFBP-1, IGFBP-3 polyclonal antibodies were from Santa Cruz Biotechnology, Inc. (CA, USA). Atlas rat cDNA arrays consist of 588 rat fragments organized into broad functional groups including housekeeping and negative control cDNAs spotted in duplicate dots were from Clontech, (Palo Alto, Calif., USA).

Animals, Histology and Cells

Male Wistar rats (200-250 gr) were fed ad libitum and received humane care under institutional guidelines. Liver fibrosis was induced by intraperitoneal administration of TAA (200 mg/kg twice weekly) for 1, 2 and 4 weeks. Halofuginone (5 ppm) was given in the diet (Nagler A. et al., 1988. Ann Surg 227:575-582; Nagler A. et al., 1999. Am J Obstet Gynecol 180:558-563; Bruck R. et al., 2001. Hepatology 33:379-386). Preparation of sections, in situ hybridization and immunohistochemistry were performed as previously described (Bruck R. et al., 2001. Hepatology 33:379-386). IGFBP-1 probe was labeled by uridine [αS³⁵]triphosphate. Cell lines used were human hepatocellular carcinoma HepG2, Hep3B and Huh-7, human fibroblasts Detroit 551, rat osteosarcoma ROS 17/2.8 and SV40-immortalized rat HSC-T6 (generously provided by Dr. S. L Friedman). Cells were grown in DMEM with 10% FCS, and the medium was replaced by serum-free DMEM after overnight plating. Following serum starvation (18 h), the medium was replaced with the fresh medium with or without halofuginone. Rat primary hepatocytes were prepared as described (Libal-Weksler Y. et al., 2001. J Nutr Biochem 12:458-464) and plated on fibronectin-coated 6-well plates at a density of 1.5×10⁶ cells/well in DMEM with 10% FCS. Cells after 18 h of seeding were serum-starved for 6 h and treated with 1 nM Halofuginone or 10 nM insulin for additional 24 h. Conditioned medium was collected and cells were scraped directly into TRI Reagent for total RNA purification. For proliferation evaluation, cells were plated in 24 well plates in DMEM with 10% FCS and direct estimation of cell number was made using cell counter.

Partial Hepatectomy

Adult male Sprague-Dawley rats (140-200 g) were maintained on rat chow and water under standard conditions. 70% partial hepatectomy (PHx) was performed according to Higgins and Anderson under light anesthesia by removing the median and left lateral lobes (Ishak K et al., 1995. J Hepatol 22(6):696-699). Animals (6 per group) were sacrificed under ether anesthesia at different intervals post operatively. Excised liver was weighed and 0.5 g samples were treated with 4% paraformaldehyde for histochemistry, immunostaining and in situ hybridization or frozen in liquid nitrogen for RNA extraction and hydroxyproline content. Liver cirrhosis was induced by intraperitoneal administration of TAA 0.2 mg/g body weight twice weekly for eight weeks. Such a procedure resulted in characteristic micronodular lesions. Halofuginone was given in the diet at concentrations of either 5 or 10 ppm.

Ishak Staging of Fibrosis

The Ishak staging system (Ishak et al., supra) was used to determine the level of fibrosis. 0—normal liver architecture; 1—Fibrosis expansion of some portal areas, with or without short fibrous septa; 2—Fibrosis expansion of most portal areas, with or without short fibrous septa; 3—Fibrous expansion of most portal areas with occasional portal to portal bridging; 4—Fibrous expansion of portal areas with marked bridging (portal to portal as well as portal to central); 5—Marked bridging (P-P and/or P-C) with occasional nodules; 6—Cirrhosis. Grading was performed following staining with Sirius red (Junquiera L C. et al., 1979. Anal Biochem. 94(1):96-99) and evaluating 10 separate fields.

Monitoring Liver Regeneration

Liver regeneration was monitored by PCNA immunostaining and by liver weight. Restituted liver mass was estimated by weighing the resected portion of the liver, which was used to calculate total pre-hepatectomy liver weight (a). Upon sacrifice, the remaining liver was excised, weighed and the respective 30% liver weight reduced (b). Restituted liver mass was expressed as percentage of the ratio of b divided by a, multiplied by 100.

RNA Purification and Atlas Rat cDNA Arrays Hybridization

Total RNA from liver tissue (5 μg comprising identical amounts of RNA from 3 rats) was isolated with TRI Reagent, treated with DNaseI and reverse transcribed in the presence of [αa-³²P]DATP (3000 Ci/mmol) using MMLV reverse transcriptase (50 U/μl) for 25 min at 48° C. Array membranes were pre-hybridized in ExpressHyb solution at 68° C. for 1 h, and hybridized with labeled cDNA probes overnight at 68° C. The second raw from bottom represents the housekeeping genes. The cDNA microarrays images were analyzed by Atlasimage 1.01 software (Clontech, USA). The background was calculated by default external background that takes into consideration the background signals and the blank space. The signal threshold was based on the background and the signal intensity was normalized globally by means of the sum method.

Immunoprecipitation, Western and Northern Blots and Probes

HepG2 conditioned medium was incubated with goat anti-IGFBP-1 or normal goat serum (1:100 dilution) overnight at 4° C. The immune complexes were precipitated by incubation with protein A-Sepharose for 2 h at 4° C. followed by centrifugation at 13,000 rpm for 5 min. The presence of IGFBP-1 protein in the supernatant and pellet was analyzed by Western blot. For Western blots, conditioned medium (45 μl) was electrophoresed on 12.5% SDS-PAGE, transferred onto nitrocellulose membranes and probed with anti-IGFBP-1. For Northern blots, 10 μg of total RNA were resolved under denaturating conditions on 1.2% agarose/formaldehyde gels, transferred onto Nytran N nylon membranes and hybridized with ³²P-labeled cDNA probe overnight at 68° C. The probes were generated by RT-PCR amplification, with the following primers pairs:

Rat IGFBP-3:
5′-CAGAGCACAGACACCCAGAA-3′
and
5′-AAATCAAGAAGGCAGAGGGC-3′
Human IGFBP-1:
5′-GCACAGGAGACATCAGGAGA-3′
and
5′-GCAACATCACCACAGGTAGC-3′
Rat IGFBP-1:
5′-CCACCACTTCCGCTACTATCT-3′
and
5′-GCTGTTCCTCTGTCATCTCTGG-3′.

Cell Motility Assay

Motility was evaluated by HitKit (Cellomics, Inc. Pittsburgh, Pa., USA). HSC were plated on a lawn of microscopic beads. As the cells move, they phagocytose and push aside the beads, clearing tracks behind them. The track area, visualized by phase contrast microscopy, is proportional to the magnitude of cell movement. Time-lapse movies were acquired at 30 minutes intervals using DeltaVision digital microscopy system and processed using the Priism software. The results are presented as the average±S.E of phagokinetic tracks (PKT) in μm² after cell area subtraction.

### Example 1

### Effect of Halofuginone on TAA-Induced Liver Fibrosis

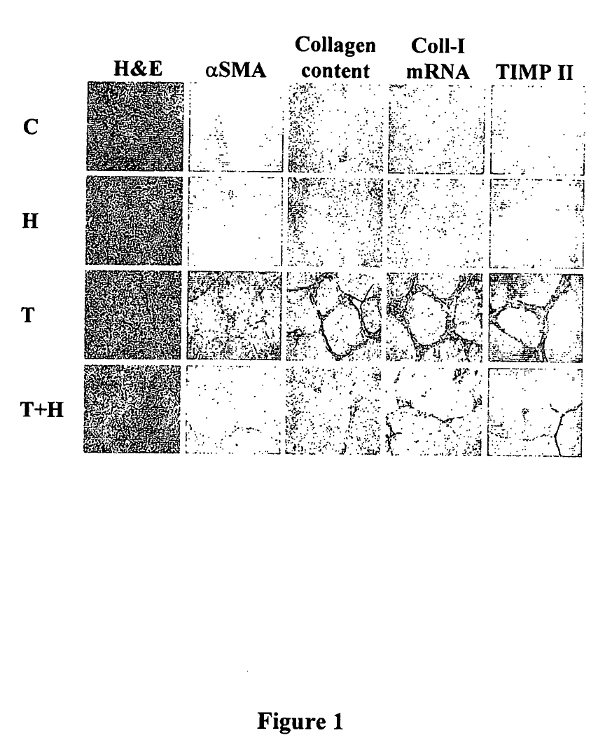

Liver sections of the control rats were devoid of ECM in general (H&E staining) and of collagen in particular (Sirius red staining). When ASMA antibodies were used, no stellate cells were detected, which suggests that the latter were in their quiescent state. No cells expressing the collagen α1(I) gene or synthesizing TIMP-2 were detected by in situ hybridization or immunohistochemistry, respectively (FIG. 1). No changes in the above parameters were observed in rats treated with halofuginone alone. When treated for 4 weeks with TAA, the livers exhibited a marked increase in ECM content, and displayed bundles of collagen that surrounded the lobules and resulted in large fibrous septa and distorted tissue architecture. These septa were populated by αSMA-positive cells expressing high levels of the collagen α1(I) gene and containing high levels of TIMP-2, all of which are characteristic of advanced fibrosis. These sections were diagnosed as grade 5-6 according to the Ishak staging system. Halofuginone given orally prevented the activation of most of the stellate cells and only traces of αSMA-positive cells were detected. The remaining stellate cells expressed low levels of collagen α1(I) gene that resulted in low levels of collagen. The level of TIMP-2 was also reduced compared with that in the TAA-treated rats. RNA from the sections that had been diagnosed as grade 1-2 according to Ishak, were used for the Atlas micro-arrays.

### Example 2

### Liver Regeneration

The halofuginone-dependent decrease in liver Ishak staging was accompanied by an improved regenerative capacity. Eight weeks of halofuginone treatment resulted in close to normal values in liver mass, significantly higher than the values recorded in the control food treated group (24.2±5.7 vs.13.7±4.5, p<0.05) (FIG. 2A). This increase was associated with PCNA labeling index of 31.4±6.4 as compared to 18.8±2.9 in untreated animals (FIG. 2B).

It is worth noting that the levels of PCNA prior to PHx varied between the above groups. PCNA staining before PHx was negligible in the healthy control group. TAA feeding was characterized as expected by a large number of proliferating cells. TAA removal either in the presence or absence of halofuginone resulted in a low labeling index despite the histopathology noted in the non-treated group. The ability of the two groups however to respond to 70% PHx was different, demonstrating a significant improved capacity to regenerate following halofuginone treatment.

### Example 3

### Halofuginone-Dependent Gene Expression

cDNA array hybridization analyses were used in an attempt to identify genes that are expressed differently in TAA-treated liver biopsies (FIG. 3A) compared with those treated with both TAA and halofuginone (FIG. 3B). A few differentially expressed genes were identified (Table 1). Some were up regulated by halofuginone (IGFBP-1; PRL-1 and Apolipoprotein A-IV) while others were down regulated (E-FABP, proteasome activator 28α, Peripheral myelin protein 22, Alcohol sulfotransferase and TIMP-2).

TABLE 1
List of differentially expressed genes

| Number | Gene | Fold change | Category |
|---|---|---|---|
| 1 | Insulin like growth factor binding | (+)2.8 | Extracellular transporters and |
| | protein 1 (IGFBP-1) | | carrier proteins |
| 2 | Protein tyrosine phosphatase 4A1 | (+)1.8 | Cell cycle |
| | (PRL-1) | | |
| 3 | Apolipoprotein A-IV (APOA-IV) | (+)1.7 | Metabolism of cofactors, vitamins |
| 4 | PI3-kinase p85- alpha subunit | (+)1.6 | Phosphoinositol kinases |
| | (PI3K) | | |
| 5 | MAPK 38 | (+)1.6 | Intracellular kinase network members |
| 6 | Proteosome component C8 | (−)2.6 | Proteosomal proteins |
| 7 | Epidermal fatty acid binding | (−)2.4 | |
| | protein (E-FABP) | | |
| 8 | SR13 myelin protein; PMP-22 | (−)2.4 | Cell surface antigens |
| 9 | PCNA | (−)2.1 | DNA polymerases, replication factors |
| 10 | Proteasome activator rPA28 | (−)2.3 | Oncogenes and tumor suppressors |
| | subunit α | | |
| 11 | c-K-ras 2b proto-oncogene | (−)2.1 | Oncogenes and tumor suppressors |
| 12 | Alcohol sulfotransferase A | (−)2.0 | Complex lipid metabolism |
| 13 | Tissie inhibitor of | (−)1.9 | Protease inhibitors |
| | metalloproteinase2 (TIMP2 | | |

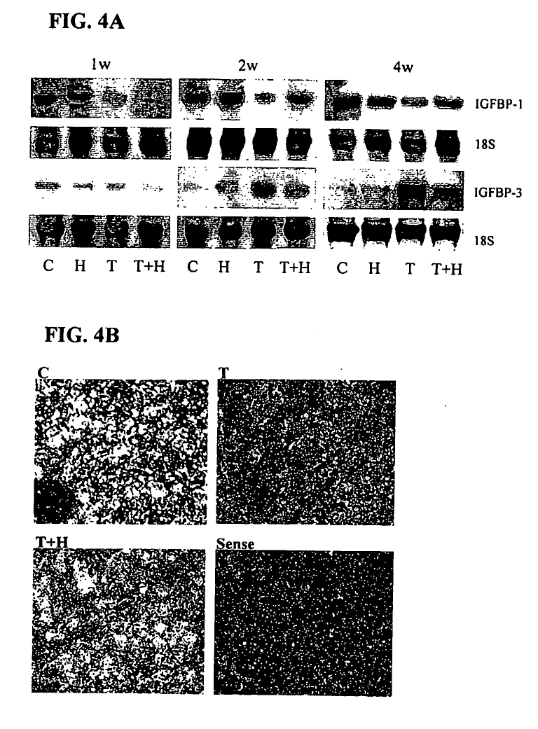

In an effort to validate the Atlas microarray results, two of the genes—PRL-1 and Apolipoprotein AIV—were analyzed by Northern blotting and the results confirmed the Atlas microarray findings (FIG. 3C). Reduction in TIMP-2 content after halofuginone treatment was also demonstrated (FIG. 1). Because of the well-documented involvement of the IGF-1 /IGFBP axis in liver fibrosis and regeneration, we focused our attention on the IGFBP-1 gene. The effect of halofuginone on the IGFBP-1 gene expression was confirmed by Northern blots analysis (FIG. 4A). After one week of TAA treatment, a reduction in the IGFBP-1 gene expression was observed without any effect of halofuginone treatment. In contrast, after 2 and 4 weeks of treatment, halofuginone prevented the TAA-induced down-regulation expression of the IGFBP-1 gene. A slight effect of halofuginone alone on the level of IGFBP-1 mRNA was observed (FIG. 4A). To determine if IGFBP-1 was the only member of the family affected by halofuginone, Northern blot analysis with IGFBP-3 probe of the same liver biopsies was performed. No changes in the IGFBP-3 mRNA levels were found in any of the groups after 1 week of treatment. After 2 and 4 weeks, TAA caused an increase in the IGFBP-3 level that was partially prevented by halofuginone. Halofuginone alone had no effect on the IGFBP-3 mRNA levels at any time-points examined. The effect of halofuginone was further confirmed by in situ hybridization (FIG. 4B). High levels of expression of the IGFBP-1 were observed in the control livers. TAA treatment caused a decrease in the expression of the IGFBP-1 gene that was prevented by halofuginone.

### Example 3

### Effect of Halofuginone on IGFBP-1 Synthesis

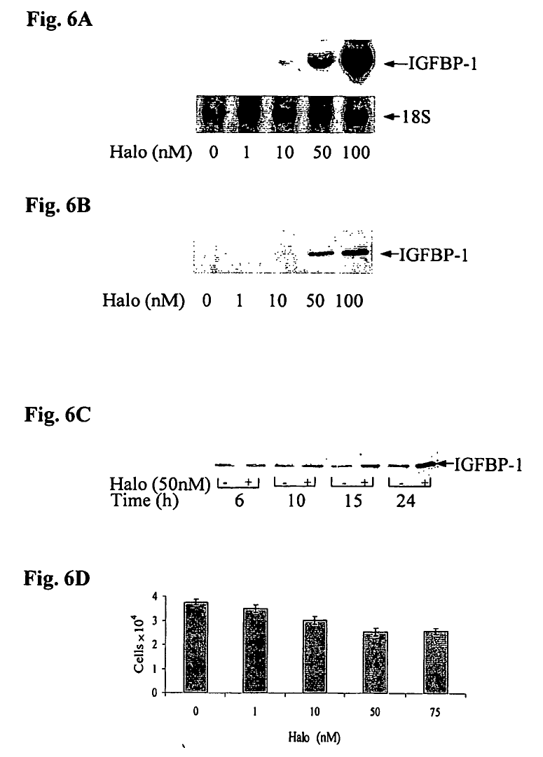

Rat primary hepatocytes, HepG2, Hep3B, Huh-7 and HSC were used to identify the source of the halofuginone-dependent synthesis of IGFBP-1. In addition, cell-lines derived from other tissues (fibroblasts and osteoblasts) were used as well. Only cells of the hepatocyte origin demonstrated increased IGFBP-1 gene expression and synthesis in response to halofuginone (FIG. 5A). In rat primary hepatocytes, insulin caused reduction in IGFBP-1 synthesis in agreement with other studies (Ishak K. et al., J Hepatol; 22:696-699) while halofuginone, at concentration as low as 1 nM, increased the synthesis of IGFBP-1 (FIG. 5B). In HepG2, no expression of the IGFBP-1 gene was detected without halofuginone (FIG. 6A). Halofuginone, at concentrations of 10 nM, increased IGFBP-1 gene expression and a further increase was observed at higher concentrations. Without halofuginone, very low (in some cases undetectable) levels of IGFBP-1 were detected in the conditioned medium of HepG2 cells (FIG. 6B). An increase in the level of IGFBP-1 was observed starting at 50 nM of halofuginone. Increased IGFBP-1 gene expression was observed as early as 6 h after halofuginone treatment (FIG. 6C) resulted in an increase in the IGFBP-1 content in the conditioned media after 10-15 h (FIG. 6D). A significant reduction in cell proliferation was observed after 24 h of incubation of HepG2 cells with halofuginone at concentration that affect IGFBP-1 synthesis (FIG. 6E). The presence of halofuginone throughout the incubation period was not essential and one hour of incubation with halofuginone was sufficient to ensure the detection of an increase in IGFBP-1 secretion 23 h later. This level of expression increased with increasing incubation time with halofuginone (FIG. 7A). During this period, de novo protein synthesis was required to demonstrate any effect of halofuginone on IGFBP- I gene expression, since incubation with cyclohexamide annulled the halofuginone-dependent increase in the IGFBP-1 gene expression (FIG. 7B).

### Example 4

### Stellate Cells Motility

HepG2 cells were incubated with 50 nM halofuginone for 11 h after which the medium was removed, the cells washed twice with DMEM to remove any traces of halofuginone and incubated with a fresh medium for additional 13 h. After halofuginone removal the cells continued to secrete IGFBP-1 and at the end of the incubation period the conditioned medium contained high levels of IGFBP-1 compare to the untreated cells (FIG. 8A). When added to HSC, the medium containing IGFBP-1 caused a significant inhibition in cell motility. Immunoprecipitation of IGFBP-1 from the condition medium abolished the inhibitory effect on HSC motility while no such effect was observed when normal serum was used (FIG. 8B).

### REFERENCES

Arthur M J, Mann D A. Iredale J P. Tissue inhibitors of metalloproteinases, hepatic stellate cells and liver fibrosis. J Gastroenterol Hepatol 1998;13:S33-S38.

Bruck R, Genina O, Aeed H, Alexiev R, Nagler A, Pines M. Halofuginone to prevent and treat thioacetamide-induced liver fibrosis in rats. Hepatology 2001;33:379-386.

Choi E T, Callow A D, Sehgal N L, Brown D M, Ryan U S. Halofuginone, a specific collagen type I inhibitor, reduces anastomotic intima hyperplasia. Arch Surg 1995;130:257-261.

Cichy S B, Uddin S, Danilkovich A, Guo S, Klippel A, Unterman T G. Protein kinase B/Akt mediates effects of insulin on hepatic insulin-like growth factor binding protein-1 gene expression through a conserved insulin response sequence. J Biol Chem 1998;273: 6482-6487.

Fan S, Ma Y X, Wang J A, Yuan R Q, Meng Q, Cao Y, Laterra J J, Goldberg I D, Rosen E M. The cytokine hepatocyte growth factor/scatter factor inhibits apoptosis and enhances DNA repair by a common mechanism involving signaling through phosphatidyl inositol 3′ kinase. Oncogene 2000;19:2212-2223.

George J, Roulot D, Koteliansky V E, Bissell D M. In vivo inhibition of rat stellate cells activation by soluble transforming growth factor beta type II receptor: a potential new therapy for hepatic fibrosis. Proc Natl Acad Sci USA 1999; 96:12719-12724.

Halevy O, Nagler A, Levi-Schaffer F, Genina O, Pines M. Inhibition of collagen type I synthesis by skin fibroblasts of graft versus host disease and scleroderma patients: effect of halofuginone. Biochem Pharmacol 1996;52:1057-1063.

Iredale J P, Benyon R C, Arthur M J, Ferris W F, Alcolado R, Winwood P, Clark N, et al. Tissue inhibitor of metalloproteinase-1 messenger RNA expression is enhanced relative to interstitial collagenase messenger RNA in experimental liver injury and fibrosis. Hepatology 1996;24:176-184.

Ishak K, Baptista A, Bianchi L, Callea F, De Groote J, Gudat F, Denk H, et al. Histological grading and staging of chronic hepatitis. J Hepatol 1995;22:696-699.

Junquiera L C, Junqueira L C, Brentani R R. A simple and sensitive method for the quantitative estimation of collagen. Anal Biochem 1979;94(1):96-99.

Levi-Schaffer F, Nagler A, Slavin S, Knopov V, Pines M. Inhibition of collagen synthesis and changes in skin morphology in murine graft versus host disease and tight skin mice: effect of halofuginone. J Invest Dermatol 1996; 106:84-88.

Libal-Weksler Y, Gotlibovitz O, Stark A H, Madar Z. Diet and diabetic state modify glycogen synthase activity and expression in rat hepatocytes. J Nutr Biochem 2001; 12:458-464.

Nagler A, Firman N, Feferman R, Cotev S, Pines M, Shoshan S. Reduction in pulmonary fibrosis in vivo by halofuginone. Am J Respir Crit Care Med 1996; 154:1082-1086.

Nagler A, Genina O, Lavelin I, Ohana M, Pines M. Halofuginone, an inhibitor of collagen type I synthesis, prevents formation of postoperative adhesions formation in the rat uterine horn model. Am J Obstet Gynecol 1999;180:558-563.

Nagler A, Rivkind A I, Raphael J, Levi-Schaffer F, Genina O, Lavelin I, Pines M. Halofuginone—an inhibitor of collagen type I synthesis—prevents post-operation abdominal adhesions formation. Ann Surg 1998;227:575-582.

Nyska M, Nyska A, Rivlin E, Porat S, Pines M, Shoshan S, Nagler A. Topically applied halofuginone, an inhibitor of collagen type I transcription, reduces peritendinous fibrous adhesions following surgery. Connect Tissue Res 1996;34:97-103.

Pines M, Domb A, Ohana M, Inbar J, Genina O, Alexiev R, Nagler A. Reduction in Dermal fibrosis in the tight-skin (Tsk) mouse after local application of halofuginone. Biochem Pharmacol 2001;62:1221-1227.

Pines M, Knopov V, Genina O, Lavelin I, Nagler A. Halofuginone, a specific inhibitor of collagen type I synthesis, prevents dimethylnitrosamine-induced liver cirrhosis. J Hepatol 1997;26: 391-398.

Spira G, Mawasi N, Paizi M, Anbinder N, Genina, O, Alexiev R, Pines M. Halofuginone, a collagen type I inhibitor improves liver regeneration in cirrhotic rats. J Hepatol 2002;37:331-339.

1. A method for improving liver regeneration comprising administering to an individual in need thereof a pharmaceutical composition comprising a therapeutically effective amount of a compound having the formula: 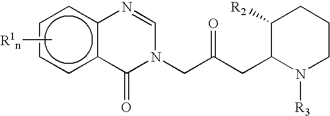
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

2. The method according to claim 1 wherein the compound is halofuginone.

3. A method for treating or preventing pathological processes related to alterations in gene expression during fibrotic processes, comprising administering to an individual in need thereof a pharmaceutical composition comprising a therapeutically effective amount of a compound having the formula: 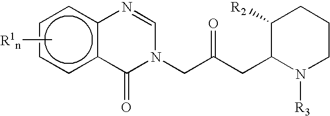
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

4. The method according to claim 3 wherein the compound is halofuginone.

5. The method according to claim 3 wherein the gene expression includes at least one gene selected from:
IGFBP-1—Insulin like growth factor binding protein 1
IGFBP-3—Insulin like growth factor binding protein 3
PRL-1 (PTP4A1)—protein tyrosine phosphatase 4A1
APO-AIV—Apolipoprotein A—IV precursor
PI 3-kinase p85-alpha subunit
MAP kinase p38—Mitogen activated protein kinase p38
Proteasome component C8
E-FABP—Epidermal fatty acid-binding protein
PMP—peripheral myelin protein (PMP-22/SR13)
PCNA—proliferation cell nuclear antigen
Proteasome activator rPA28 subunit alpha
c-K-ras 2b proto-oncogene
ST2A2—Alcohol sulfotransferase A, Probable alcohol sulfotransferase
TIMP-2—Metalloproteinase inhibitor 2 (Precursor), Tissue inhibitor of metalloproteinase 2
MMP-3—metalloproteinase 3
MMP-13—metalloproteinase 13

6. The method according to claim 3 wherein the gene is a member of the IGFBP family.

7. The method according to claim 6 wherein the gene is IGFBP-1.

8. The method according to claim 5 wherein the gene is IGFBP-3.

9. The method according to claim 3 wherein the fibrotic process is liver fibrosis.

10. A method for treating or preventing pathological processes related to toxin induced alterations in gene expression comprising administering to an individual in need thereof a pharmaceutical composition comprising a therapeutically effective amount of a compound having the formula: 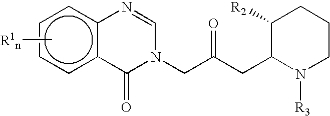
wherein: n=1 -2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

11. The method of claim 10 wherein the toxin is thioacetamide (TAA).

12. The method according to claim 10 wherein the compound is halofuginone.

13. The method according to claim 10 wherein the gene expression includes at least one gene selected from:
IGFBP-1—Insulin like growth factor binding protein 1
IGFBP-3—Insulin like growth factor binding protein 3
PRL-1 (PTP4A1)—protein tyrosine phosphatase 4A1
APO-AIV—Apolipoprotein A—IV precursor
PI 3-kinase p85-alpha subunit
MAP kinase p38—Mitogen activated protein kinase p38
Proteasome component C8
E-FABP—Epidermal fatty acid-binding protein
PMP—peripheral myelin protein (PMP-22/SR13)
PCNA—proliferation cell nuclear antigen
Proteasome activator rPA28 subunit alpha
c-K-ras 2b proto-oncogene
ST2A2—Alcohol sulfotransferase A, Probable alcohol sulfotransferase
TIMP-2—Metalloproteinase inhibitor 2 (Precursor), Tissue inhibitor of metalloproteinase 2
MMP-3 —metalloproteinase 3
MMP-13 —metalloproteinase 13

14. The method according to claim 10 wherein the gene is a member of the IGFBP family.

15. The method according to claim 14 wherein the gene is IGFBP-1.

16. The method according to claim 14 wherein the gene is IGFBP-3.

17. A method for treating hepatic cirrhosis by increasing the IGFBP-1 expression in hepatocyte cells comprising administering a pharmaceutical composition comprising a therapeutically effective amount of a compound having the formula: 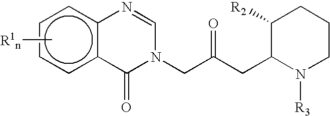
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

18. The method according to claim 17 wherein the compound is halofuginone.

19. A method for improving liver regeneration by increasing the IGFBP-1 expression in hepatocyte cells comprising administering a pharmaceutical composition comprising a therapeutically effective amount of a compound having the formula: 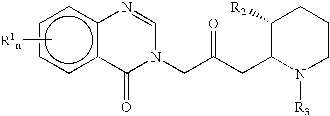
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl, and pharmaceutically acceptable salts thereof.

20. The method according to claim 19 wherein the compound is halofuginone.

21. A method for improving the capacity of a cirrhotic liver to regenerate following partial hepatectomy by inducing gene expression of at least one gene selected from IGFBP-1, PRL-1, MMP-3 and MMP-13 comprising administering a pharmaceutical composition comprising a therapeutically effective amount of a compound having the formula: 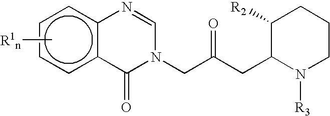
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; and pharmaceutically acceptable salts thereof.

22. The method according to claim 21 wherein the compound is halofuginone.

23. A method for improving the capacity of a cirrhotic liver to regenerate following partial hepatectomy by affecting the molecules in the signal transduction pathway of hepatocyte growth factor (HGF), comprising administering to an individual in need thereof a pharmaceutical composition comprising a therapeutically effective amount of a compound having the formula: 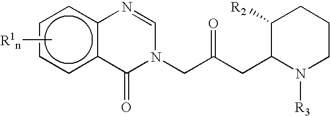
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; and pharmaceutically acceptable salts thereof.

24. The method according to claim 23 wherein the compound is halofuginone.

25. A method for increasing the amount of biologically active IGF-1, comprising administering to an individual a pharmaceutical composition comprising a therapeutically effective amount of a compound of the general formula: 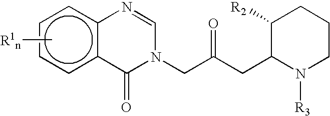
wherein: n=1-2
R₁ is at each occurrence independently selected from the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; and pharmaceutically acceptable salts thereof.

26. The method according to claim 25 wherein the compound is halofuginone.
